(19) United States
(12) Patent Application Publication
(10) Pub. No.: US 2026/0000894 A1
(43) Pub. Date: Jan. 1, 2026

(54) MONITORING CLOSED-LOOP NEURAL STIMULATION THERAPY

(71) Applicant: Saluda Medical Pty Limited, Macquarie Park, New South Wales (AU)

(72) Inventors: Dean Michael Karantonis, Macquarie Park (AU); James Hamilton Wah, Macquarie Park (AU); Peter Scott Vallack Single, Macquarie Park (AU); Daniel John Parker, Macquarie Park (AU)

(21) Appl. No.: 18/879,514

(22) PCT Filed: Jun. 30, 2023
(86) PCT No.: PCT/AU2023/050613

§ 371 Date: Dec. 27, 2024

(30) Foreign Application Priority Data
Jul. 1, 2022 (AU) 2022901856

(51) Int. Cl. A61N 1/36; A61N 1/372

(52) U.S. Cl. CPC A61N 1/36139; A61N 1/36132; A61N 1/3615; A61N 1/36171; A61N 1/37247; A61N 1/37241

(57) ABSTRACT

An implantable neuromodulation device (100) that includes a plurality of stimulus electrodes, measurement electrodes, a stimulus source and measurement circuitry (128). The device also includes a control unit (116) configured to: control the stimulus source to provide a neural stimulus to a neural pathway (180) according to a stimulus intensity parameter; measure an intensity of the evoked neural response in captured signal windows; determine a feedback variable from the measured intensity of the evoked neural response; implement a feedback loop by using the feedback variable to control the stimulus intensity parameter so as to maintain the feedback variable at a target value; convert (1404) a loop variable of the feedback loop to a representation, where the representation is one of a time-domain representation and a frequency-domain representation; and analyse (1406) the representation to determine a loop stability measure of the feedback loop.

The present application claims priority from Australian Provisional Patent Application No 2022901856 filed on 1 Jul. 2022, the contents of which are incorporated herein by reference in their entirety.

### TECHNICAL FIELD

The present invention relates to neural stimulation therapy and in particular to monitoring the stability of closed-loop neural stimulation therapy.

### BACKGROUND OF THE INVENTION

There are a range of situations in which it is desirable to apply neural stimuli in order to alter neural function, a process known as neuromodulation. For example, neuromodulation is used to treat a variety of disorders including chronic neuropathic pain, Parkinson's disease, and migraine. A neuromodulation device applies an electrical pulse (stimulus) to neural tissue (fibres, or neurons) in order to generate a therapeutic effect. In general, the electrical stimulus generated by a neuromodulation device evokes a neural response known as an action potential in a neural fibre which then has either an inhibitory or excitatory effect. Inhibitory effects can be used to modulate an undesired process such as the transmission of pain, or excitatory effects may be used to cause the desired effect such as the contraction of a muscle.

When used to relieve neuropathic pain originating in the trunk and limbs, the electrical pulse is applied to the dorsal column (DC) of the spinal cord, a procedure referred to as spinal cord stimulation (SCS). Such a device typically comprises an implanted electrical pulse generator, and a power source such as a battery that may be transcutaneously rechargeable by wireless means, such as inductive transfer. An electrode array is connected to the pulse generator, and is implanted adjacent the target neural fibre(s) in the spinal cord, typically in the dorsal epidural space above the dorsal column. An electrical pulse of sufficient intensity applied to the target neural fibres by a stimulus electrode causes the depolarisation of neurons in the fibres, which in turn generates an action potential in the fibres. Action potentials propagate along the fibres in orthodromic (in afferent fibres this means towards the head, or rostral) and antidromic (in afferent fibres this means towards the cauda, or caudal) directions. Action potentials propagating along Aβ (A-beta) fibres being stimulated in this way inhibit the transmission of pain from a region of the body innervated by the target neural fibres (the dermatome) to the brain. To sustain the pain relief effects, stimuli are applied repeatedly, for example at a frequency in the range of 30 Hz-100 Hz.

For effective and comfortable neuromodulation, it is necessary to maintain stimulus intensity above a recruitment threshold. Stimuli below the recruitment threshold will fail to recruit sufficient neurons to generate action potentials with a therapeutic effect. In almost all neuromodulation applications, response from a single class of fibre is desired, but the stimulus waveforms employed can evoke action potentials in other classes of fibres which cause unwanted side effects. In pain relief, it is therefore desirable to apply stimuli with intensity below a discomfort threshold, above which uncomfortable or painful percepts arise due to over-recruitment of Aβ fibres. When recruitment is too large, Aβ fibres produce uncomfortable sensations. Stimulation at high intensity may even recruit Aδ (A-delta) fibres, which are sensory nerve fibres associated with acute pain, cold and heat sensation. It is therefore desirable to maintain stimulus intensity within a therapeutic range between the recruitment threshold and the discomfort threshold.

The task of maintaining appropriate neural recruitment is made more difficult by electrode migration (change in position over time) and/or postural changes of the implant recipient (patient), either of which can significantly alter the neural recruitment arising from a given stimulus, and therefore the therapeutic range. There is room in the epidural space for the electrode array to move, and such array movement from migration or posture change alters the electrode-to-fibre distance and thus the recruitment efficacy of a given stimulus. Moreover, the spinal cord itself can move within the cerebrospinal fluid (CSF) with respect to the dura. During postural changes, the amount of CSF and/or the distance between the spinal cord and the electrode can change significantly. This effect is so large that postural changes alone can cause a previously comfortable and effective stimulus regime to become either ineffectual or painful.

Attempts have been made to address such problems by way of feedback or closed-loop control, such as using the methods set forth in International Patent Publication No. WO2012/155188 by the present applicant. Feedback control seeks to compensate for relative nerve/electrode movement by controlling the intensity of the delivered stimuli so as to maintain a substantially constant neural recruitment. The intensity of a neural response evoked by a stimulus may be used as a feedback variable representative of the amount of neural recruitment. A signal representative of the neural response may be sensed by a measurement electrode in electrical communication with the recruited neural fibres, and processed to obtain the feedback variable. Based on the response intensity, the intensity of the applied stimulus may be adjusted to maintain the response intensity within a therapeutic range.

It is therefore desirable to accurately measure the intensity and other characteristics of a neural response evoked by the stimulus. The action potentials generated by the depolarisation of a large number of fibres by a stimulus sum to form a measurable signal known as an evoked compound action potential (ECAP). Accordingly, an ECAP is the sum of responses from a large number of single fibre action potentials. The ECAP generated from the depolarisation of a group of similar fibres may be measured at a measurement electrode as a positive peak potential, then a negative peak, followed by a second positive peak. This morphology is caused by the region of activation passing the measurement electrode as the action potentials propagate along the individual fibres.

Approaches proposed for obtaining a neural response measurement are described by the present applicant in International Patent Publication No. WO2012/155183, the content of which is incorporated herein by reference.

However, neural response measurement can be a difficult task as a neural response component in the sensed signal will typically have a maximum amplitude in the range of microvolts. In contrast, a stimulus applied to evoke the response is typically several volts, and manifests in the measured response as crosstalk of that magnitude. Moreover, stimulus generally results in electrode artefact, which manifests in the measured response as a decaying output of the order of several millivolts after the end of the stimulus. As the neural response can be contemporaneous with the stimulus crosstalk and/or the stimulus artefact, neural response measurements present a difficult challenge of measurement amplifier design. For example, to resolve a 10 μV ECAP with 1 μV resolution in the presence of stimulus crosstalk of 5 V requires an amplifier with a dynamic range of 134 dB, which is impractical in implantable devices. In practice, many non-ideal aspects of a circuit lead to artefact, and as these aspects mostly result a time-decaying artefact waveform of positive or negative polarity, their identification and elimination can be laborious.

Evoked neural responses are less difficult to detect when they appear later in time than the artefact, or when the signal-to-noise ratio is sufficiently high. The artefact is often restricted to a time of 1-2 ms after the stimulus and so, provided the neural response is detected after this time window, a neural response measurement can be more easily obtained. This is the case in surgical monitoring where there are large distances (e.g. more than 12 cm for nerves conducting at 60 ms⁻¹) between the stimulus and measurement electrodes so that the propagation time from the stimulus site to the measurement electrodes exceeds 2 ms, which is longer than the typical duration of stimulus artefact.

However, to characterise the responses from the dorsal column, high stimulation currents are required. Similarly, any implanted neuromodulation device will necessarily be of compact size, so that for such devices to monitor the effect of applied stimuli, the stimulus electrode(s) and measurement electrode(s) will necessarily be in close proximity. In such situations the measurement process must overcome artefact directly.

Closed-loop neural stimulation therapy is governed by a number of parameters to which values must be assigned to implement the therapy. Closed-loop SCS systems need to be programmed per patient, in particular, to set an appropriate controller gain. The loop can show some instability such as ringing behaviour (under-damping) if the controller gain is set too high during programming. Even a loop that appears stable with the patient in one posture may become unstable when the patient is in another posture.

Further, once the patient is out of the clinic, circumstances can change, for example, lead migration, scar tissue accretion that could cause instability in a previously stable control loop.

Loop instability could potentially lead to uncomfortable or even painful stimulations for the patient. Further, an unstable loop could provide therapy outside the therapeutic window hence leading to a lack of efficacy in pain relief.

Further, loop instability may not be addressed by simply turning down the target response intensity via the remote control, which is often the only control the patient has over therapy apart from turning the device off entirely.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

In this specification, a statement that an element may be “at least one of” a list of options is to be understood to mean that the element may be any one of the listed options, or may be any combination of two or more of the listed options.

### SUMMARY OF THE INVENTION

Disclosed herein are systems and methods for analysing loop variables, which include stimulus intensities and/or the intensities of evoked neural responses, to determine a measure of loop stability of a closed-loop neural stimulation system. In an implementation, the loop variables may be analysed using at least one of a time-domain representation and a frequency-domain representation to determine the measure of loop stability. Appropriate actions may be taken based on the measure of loop stability to improve the loop stability.

According to a first aspect of the present technology, there is provided an implantable neuromodulation device including a plurality of electrodes including one or more stimulus electrodes and one or more measurement electrodes. The device also includes a stimulus source configured to provide neural stimuli to be delivered via the one or more stimulus electrodes to a neural pathway of a patient in order to evoke a neural response on the neural pathway. The device also includes measurement circuitry configured to capture signal windows sensed on the neural pathway via the one or more measurement electrodes subsequent to respective neural stimuli. The device also includes a control unit configured to: control the stimulus source to provide a neural stimulus according to a stimulus intensity parameter; measure an intensity of the evoked neural response in the captured signal window subsequent to the provided neural stimulus; determine a feedback variable from the measured intensity of the evoked neural response; implement a feedback loop by using the feedback variable to control the stimulus intensity parameter so as to maintain the feedback variable at a target value; convert a loop variable of the feedback loop to a representation, where the representation is one of a time-domain representation and a frequency-domain representation; and analyse the representation to determine a loop stability measure of the feedback loop.

Implementations of the first aspect may include one or more of the following features. The loop variable includes one of: the feedback variable; an error value between the feedback variable and the target value; and the stimulus intensity parameter. The representation is a time-domain representation. The time-domain representation is a histogram. The control unit is configured to analyse the representation by determining a measure of bimodality of the histogram as the loop stability measure. The control unit is further configured to take one or more actions based on the loop stability measure. The control unit is further configured to take one or more actions based on a comparison of the loop stability measure and a predetermined threshold. The one or more actions may include one or more of: adjusting one or more program parameters of the feedback loop; issuing an alert; transitioning to an open-loop operation of the implantable neuromodulation device; and shutting down the implantable neuromodulation device. The time-domain representation is a Lissajous figure. The control unit is configured to analyse the representation by determining a measure of bilobularity of the Lissajous figure as the loop stability measure. The time-domain representation is a time series. The control unit is configured to analyse the representation using a finite impulse response filter to detect an oscillation in the time series at a predetermined frequency. The predetermined frequency is half of a frequency of the provided neural stimuli. The control unit is further configured to introduce a perturbation to the feedback loop before the converting. The perturbation is a step. The control unit is configured to analyse the representation by determining a measure of under-dampedness of the time series as the loop stability measure. The control unit is configured to analyse the representation by determining a ratio of a standard deviation of stimulus intensity noise to a therapeutic range. The control unit is configured to analyse the representation by determining a ratio of a standard deviation of noise of the feedback variable to a standard deviation of noise of the feedback variable in open-loop mode. The representation is a frequency domain representation. The representation is a Fourier spectrum of the loop variable. The control unit is configured to analyse the representation by determining the magnitude of the Fourier spectrum at a predetermined frequency as the loop stability measure. The predetermined frequency is half of a frequency of the provided neural stimuli.

According to a second aspect, there is disclosed a method of determining a measure of stability of a closed-loop neural stimulation system. The method includes delivering a neural stimulus to a neural pathway of a patient in order to evoke a neural response on the neural pathway, the neural stimulus being delivered according to a stimulus intensity parameter. The method also includes capturing a signal window sensed on the neural pathway subsequent to the delivered neural stimulus. The method also includes measuring an intensity of the neural response evoked by the delivered neural stimulus in the captured signal window. The method also includes determining, from the measured intensity of the evoked neural response, a feedback variable. The method also includes implementing a feedback loop by using the determined feedback variable to control the stimulus intensity parameter so as to maintain the feedback variable at a target value. The method also includes converting a loop variable of the feedback loop to a representation, where the representation is one of a time-domain representation and a frequency-domain representation. The method also includes analysing the representation to determine a loop stability measure of the feedback loop.

Implementations of the second aspect may include one or more of the following features. The loop variable includes one of: the feedback variable; an error value between the feedback variable and the target value; and the stimulus intensity parameter. The representation is a time-domain representation. The time-domain representation is a histogram. The method includes taking one or more actions based on the loop stability measure. The time-domain representation is a Lissajous figure. The time-domain representation is a time series. The representation is a frequency domain representation.

According to a third aspect, there is disclosed a closed-loop neural stimulation system. The closed-loop neural stimulation system includes a plurality of electrodes including one or more stimulation electrodes and one or more measurement electrodes. The system also includes a stimulus source configured to provide neural stimuli to be delivered via the one or more stimulation electrodes to a neural pathway of a patient in order to evoke neural responses on the neural pathway. The system also includes measurement circuitry configured to capture signal windows sensed on the neural pathway via the one or more measurement electrodes subsequent to respective neural stimuli. The system also includes a control unit configured to: control the stimulus source to provide a neural stimulus according to a stimulus intensity parameter, measure an intensity of the evoked neural response in the captured signal window subsequent to the provided neural stimulus, determine a feedback variable from the measured intensity of the evoked neural response, implement a feedback loop by using the feedback variable to control the stimulus intensity parameter so as to maintain the feedback variable at a target value. The system also includes a processor configured to: convert a loop variable of the feedback loop to a representation, where the representation is one of a time-domain representation and a frequency-domain representation; and analyse the representation to determine a loop stability measure of the feedback loop.

Implementations of the third aspect may include one or more of the following features. The system includes an external device in communication with the control unit. The processor is part of the external device. The external device further includes a display. The processor is configured to render a user interface on the display. The user interface includes a control configured to allow a user to select the loop variable from among a plurality of candidate loop variables. The user interface includes a control configured to allow a user to select a domain of the representation from among a plurality of candidate domains. The processor is further configured to display the loop stability measure on the user interface. The processor is further configured to display the representation on the user interface.

References herein to estimation, determination, comparison and the like are to be understood as referring to an automated process carried out on data by a processor operating to execute a predefined procedure suitable to effect the described estimation, determination and/or comparison step(s). The technology disclosed herein may be implemented in hardware (e.g., using digital signal processors, application specific integrated circuits (ASICs) or field programmable gate arrays (FPGAs)), or in software (e.g., using instructions tangibly stored on non-transitory computer-readable media for causing a data processing system to perform the steps described herein), or in a combination of hardware and software. The disclosed technology can also be embodied as computer-readable code on a computer-readable medium. The computer-readable medium can include any data storage device that can store data which can thereafter be read by a computer system. Examples of the computer-readable medium include read-only memory (“ROM”), random-access memory (“RAM”), magnetic tape, optical data storage devices, flash storage devices, or any other suitable storage devices. The computer-readable medium can also be distributed over network-coupled computer systems so that the computer-readable code is stored and/or executed in a distributed fashion.

### BRIEF DESCRIPTION OF THE DRAWINGS

One or more implementations of the invention will now be described with reference to the accompanying drawings, in which:

FIG. 1 schematically illustrates an implanted spinal cord stimulator, according to one implementation of the present technology;

FIG. 2 is a block diagram of the stimulator of FIG. 1;

FIG. 3 is a schematic illustrating interaction of the implanted stimulator of FIG. 1 with a nerve;

FIG. 4a illustrates an idealised activation plot for one posture of a patient undergoing neural stimulation;

FIG. 4b illustrates the variation in the activation plots with changing posture of the patient;

FIG. 5 is a schematic illustrating elements and inputs of a closed-loop neural stimulation (CLNS) system, according to one implementation of the present technology;

FIG. 6 illustrates the typical form of an electrically evoked compound action potential (ECAP) of a healthy subject;

FIG. 7 is a block diagram of a neural stimulation therapy system including the implanted stimulator of FIG. 1 according to one implementation of the present technology;

FIG. 8 is a block diagram illustrating the data flow of a neural stimulation therapy system such as the system of FIG. 7;

FIG. 9 illustrates a generic step response of a closed-loop control system;

FIG. 10 illustrates an exemplary loop oscillation for different gain values on a fixed target value;

FIG. 11 illustrates a filter that is configured to detect loops that are unstable, according to an implementation;

FIG. 12 illustrates a histogram with a bimodal distribution indicating an unstable loop;

FIGS. 13A and 13B illustrate open loop and closed loop performance at various frequencies and gain values, according to an implementation;

FIG. 14 illustrates a flow chart pertaining to the method steps for assessing the stability of a loop, in accordance with an implementation;

FIG. 15 illustrates a flowchart pertaining to the method steps of using a histogram for assessing the stability of the loop operation; and

FIG. 16 illustrates a user interface for assessing the loop variables and displaying a loop stability measure.

### DETAILED DESCRIPTION OF THE PRESENT TECHNOLOGY

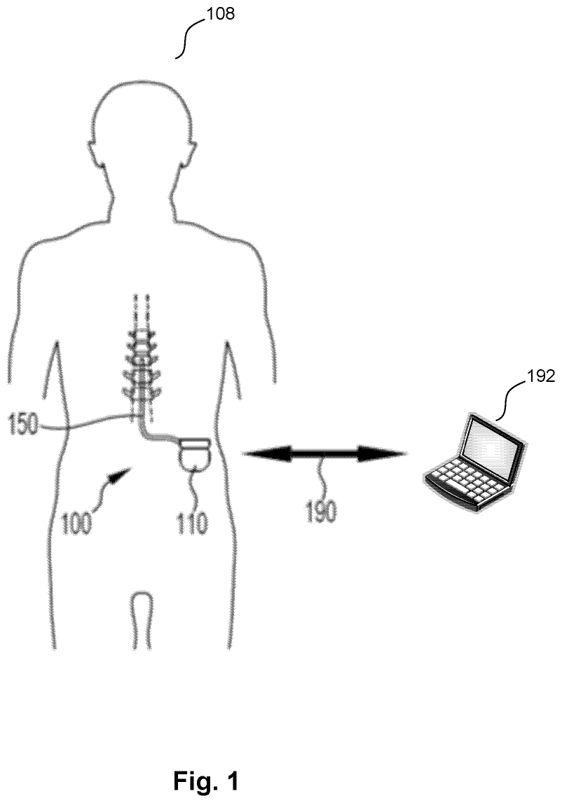

FIG. 1 schematically illustrates an implanted spinal cord stimulator 100 in a patient 108, according to one implementation of the present technology. Stimulator 100 comprises an electronics module 110 implanted at a suitable location. In one implementation, stimulator 100 is implanted in the patient's lower abdominal area or posterior superior gluteal region. In other implementations, the electronics module 110 is implanted in other locations, such as in a flank or sub-clavicularly. Stimulator 100 further comprises an electrode array 150 implanted within the epidural space and connected to the module 110 by a suitable lead. The electrode array 150 may comprise one or more electrodes such as electrode pads on a paddle lead, circular (e.g., ring) electrodes surrounding the body of the lead, conformable electrodes, cuff electrodes, segmented electrodes, or any other type of electrodes capable of forming unipolar, bipolar or multipolar electrode configurations for stimulation and measurement. The electrodes may pierce or affix directly to the tissue itself.

Numerous aspects of the operation of implanted stimulator 100 may be programmable by an external computing device 192, which may be operable by a user such as a clinician or the patient 108. Moreover, implanted stimulator 100 serves a data gathering role, with gathered data being communicated to external device 192 via a transcutaneous communications channel 190. Communications channel 190 may be active on a substantially continuous basis, at periodic intervals, at non-periodic intervals, or upon request from the external device 192. External device 192 may thus provide a clinical interface configured to program the implanted stimulator 100 and recover data stored on the implanted stimulator 100. This configuration is achieved by program instructions collectively referred to as the Clinical Programming Application (CPA) and stored in an instruction memory of the clinical interface.

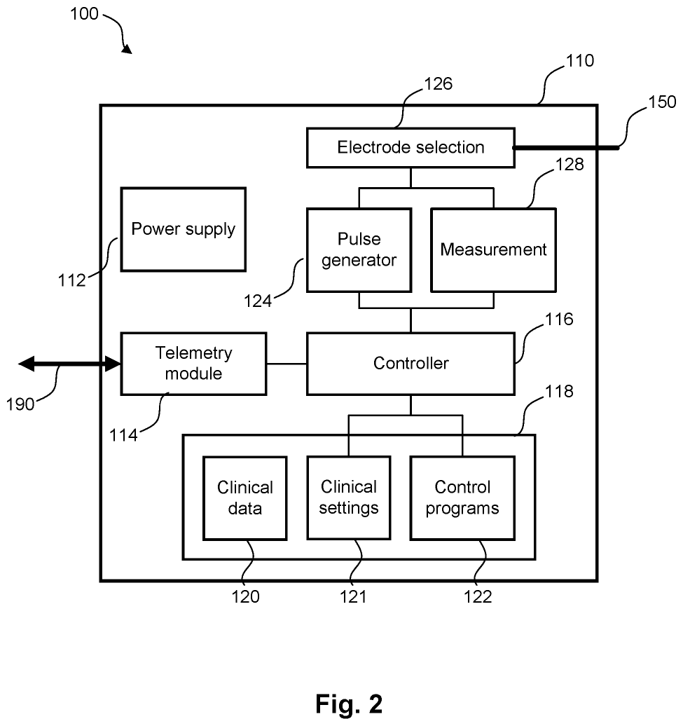

FIG. 2 is a block diagram of the stimulator 100. Electronics module 110 contains a battery 112 and a telemetry module 114. In implementations of the present technology, any suitable type of transcutaneous communications channel 190, such as infrared (IR), radiofrequency (RF), capacitive and/or inductive transfer, may be used by telemetry module 114 to transfer power and/or data to and from the electronics module 110 via communications channel 190. Module controller 116 has an associated memory 118 storing one or more of clinical data 120, clinical settings 121, control programs 122, and the like. Controller 116 is configured by control programs 122, sometimes referred to as firmware, to control a simulation source, such as a pulse generator 124 to generate stimuli, such as in the form of electrical pulses, in accordance with the clinical settings 121 and control programs 122. Electrode selection module 126 switches the generated pulses to the selected electrode(s) of electrode array 150, for delivery of the pulses to the tissue surrounding the selected electrode(s). Measurement circuitry 128, which may comprise an amplifier and/or an analog-to-digital converter (ADC), is configured to process signals comprising neural responses sensed at measurement electrode(s) of the electrode array 150 as selected by electrode selection module 126.

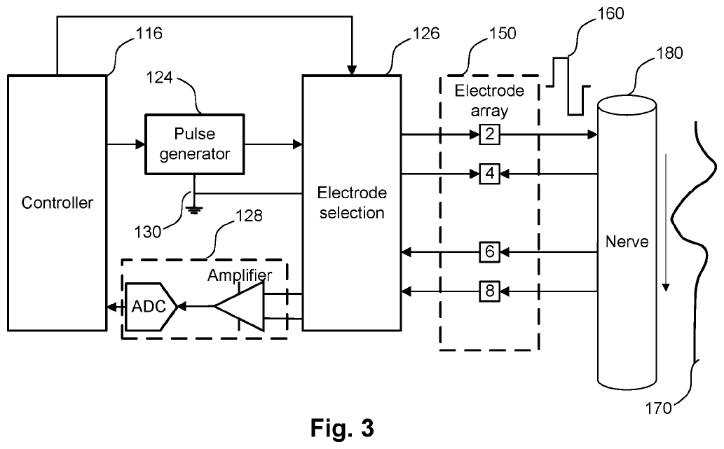

FIG. 3 is a schematic illustrating interaction of the implanted stimulator 100 with a nerve 180 in the patient 108. In the implementation illustrated in FIG. 3 the nerve 180 may be located in the spinal cord, however in alternative implementations the stimulator 100 may be positioned adjacent any desired neural tissue including a peripheral nerve, visceral nerve, parasympathetic nerve or a brain structure. Electrode selection module 126 selects a stimulus electrode 2 of electrode array 150 through which to deliver a pulse from the pulse generator 124 to surrounding tissue including nerve 180. A pulse may comprise one or more phases, e.g. a biphasic stimulus pulse 160 comprises two phases. Electrode selection module 126 also selects a return electrode 4 of the electrode array 150 for stimulus current return in each phase, to maintain a zero net charge transfer. An electrode may act as both a stimulus electrode and a return electrode over a complete multiphasic stimulus pulse. The use of two electrodes in this manner for delivering and returning current in each stimulus phase is referred to as bipolar stimulation. Alternative embodiments may apply other forms of bipolar stimulation, or may use a greater number of stimulus and/or return electrodes. The set of stimulus and return electrodes and their respective polarities is referred to as the stimulus electrode configuration. Electrode selection module 126 is illustrated as connecting to a ground 130 of the pulse generator 124 to enable stimulus current return via the return electrode 4. However, other connections for currently return may be used in other implementations.

Delivery of an appropriate stimulus via stimulus electrodes 2 and 4 to the nerve 180 evokes a neural response 170 comprising an evoked compound action potential (ECAP) which will propagate along the nerve 180 as illustrated at a rate known as the conduction velocity. The ECAP may be evoked for therapeutic purposes, which in the case of a spinal cord stimulator for chronic pain may be to create paraesthesia at a desired location. To this end, the stimulus electrodes 2 and 4 are used to deliver stimuli periodically at any therapeutically suitable frequency, for example 30 Hz, although other frequencies may be used including frequencies as high as the kHz range. In alternative implementations, stimuli may be delivered in a non-periodic manner such as in bursts, or sporadically, as appropriate for the patient 108. To program the stimulator 100 to the patient 108, a clinician may cause the stimulator 100 to deliver stimuli of various configurations which seek to produce a sensation that is experienced by the user as paraesthesia. When a stimulus electrode configuration is found which evokes paraesthesia in a location and of a size which is congruent with the area of the patient's body affected by pain of a quality that is comfortable for the patient, the clinician nominates that configuration for ongoing use. The program parameters may be loaded into the memory 118 of the stimulator 100 as the clinical settings 121.

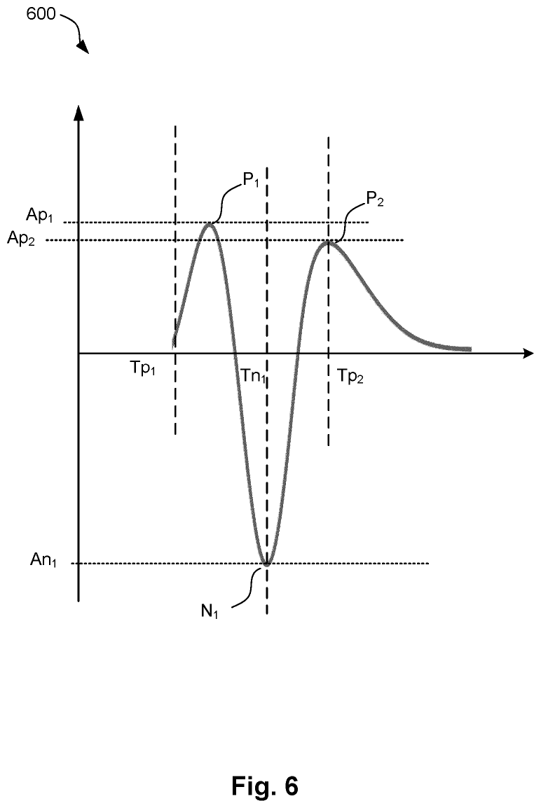

FIG. 6 illustrates the typical form of an ECAP 600 of a healthy subject, as recorded at a single measurement electrode referenced to the system ground 130. The shape and duration of the single-ended ECAP 600 shown in FIG. 6 is predictable because it is a result of the ion currents produced by the ensemble of fibres depolarising and generating action potentials (APs) in response to stimulation. The evoked action potentials (EAPs) generated synchronously among a large number of fibres sum to form the ECAP 600. The ECAP 600 generated from the synchronous depolarisation of a group of similar fibres comprises a positive peak P1, then a negative peak N1, followed by a second positive peak P2. This shape is caused by the region of activation passing the measurement electrode as the action potentials propagate along the individual fibres.

The ECAP may be recorded differentially using two measurement electrodes, as illustrated in FIG. 3. Differential ECAP measurements are less subject to common-mode noise on the surrounding tissue than single-ended ECAP measurements. Depending on the polarity of recording, a differential ECAP may take an inverse form to that shown in FIG. 6, i.e. a form having two negative peaks N1 and N2, and one positive peak P1. Alternatively, depending on the distance between the two measurement electrodes, a differential ECAP may resemble the time derivative of the ECAP 600, or more generally the difference between the ECAP 600 and a time-delayed copy thereof.

The ECAP 600 may be characterised by any suitable characteristic(s) of which some are indicated in FIG. 6. The amplitude of the positive peak P1 is Ap₁ and occurs at time Tp₁. The amplitude of the positive peak P2 is Ap₂ and occurs at time Tp₂. The amplitude of the negative peak P1 is An₁ and occurs at time Tn₁. The peak-to-peak amplitude is Ap₁+An₁. A recorded ECAP will typically have a maximum peak-to-peak amplitude in the range of microvolts and a duration of 2 to 3 ms.

The stimulator 100 is further configured to detect the existence and measure the intensity of ECAPs 170 propagating along nerve 180, whether such ECAPs are evoked by the stimulus from electrodes 2 and 4, or otherwise evoked. To this end, any electrodes of the array 150 may be selected by the electrode selection module 126 to serve as recording electrode 6 and reference electrode 8, whereby the electrode selection module 126 selectively connects the chosen electrodes to the inputs of the measurement circuitry 128. Thus, signals sensed by the measurement electrodes 6 and 8 subsequent to the respective stimuli are passed to the measurement circuitry 128, which may comprise a differential amplifier and an analog-to-digital converter (ADC), as illustrated in FIG. 3. The recording electrode and the reference electrode are referred to as the measurement electrode configuration. The measurement circuitry 128 for example may operate in accordance with the teachings of the above-mentioned International Patent Publication No. WO2012/155183.

Signals sensed by the measurement electrodes 6, 8 and processed by measurement circuitry 128 are further processed by an ECAP detector implemented within controller 116, configured by control programs 122, to obtain information regarding the effect of the applied stimulus upon the nerve 180. In some implementations, the sensed signals are processed by the ECAP detector in a manner which measures and stores one or more characteristics from each evoked neural response or group of evoked neural responses contained in the sensed signal. In one such implementation, the characteristics comprise a peak-to-peak ECAP amplitude in microvolts (μV). For example, the sensed signals may be processed by the ECAP detector to determine the peak-to-peak ECAP amplitude in accordance with the teachings of International Patent Publication No. WO2015/074121, the contents of which are incorporated herein by reference. Alternative implementations of the ECAP detector may measure and store an alternative characteristic from the neural response, or may extract and store two or more characteristics from the neural response.

Stimulator 100 applies stimuli over a potentially long period such as days, weeks, or months and during this time may store characteristics of neural responses, clinical settings, paraesthesia target level, and other operational parameters in memory 118. To effect suitable SCS therapy, stimulator 100 may deliver tens, hundreds or even thousands of stimuli per second, for many hours each day. Each neural response or group of responses generates one or more characteristics such as a measure of the intensity of the neural response. Stimulator 100 thus may produce such data at a rate of tens or hundreds of Hz, or even kHz, and over the course of hours or days this process results in large amounts of clinical data 120 which may be stored in the memory 118. Memory 118 is however necessarily of limited capacity and care is thus required to select compact data forms for storage into the memory 118, to ensure that the memory 118 is not exhausted before such time that the data is expected to be retrieved wirelessly by external device 192, which may occur only once or twice a day, or less.

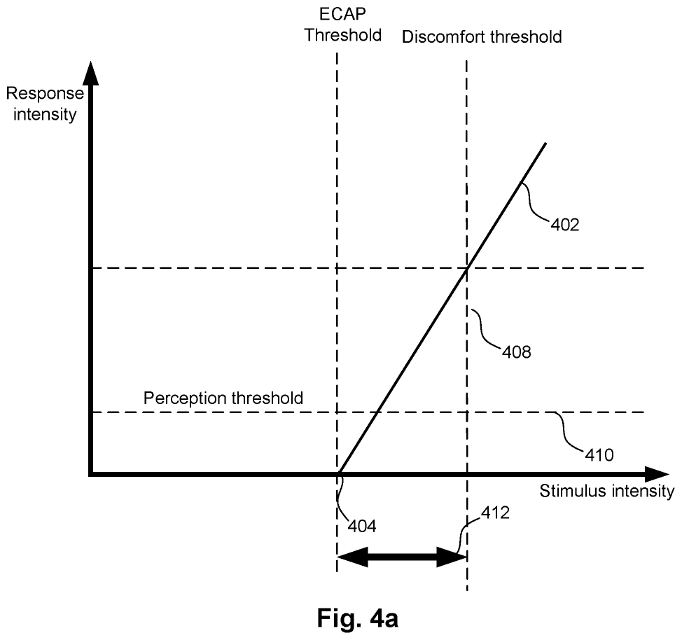

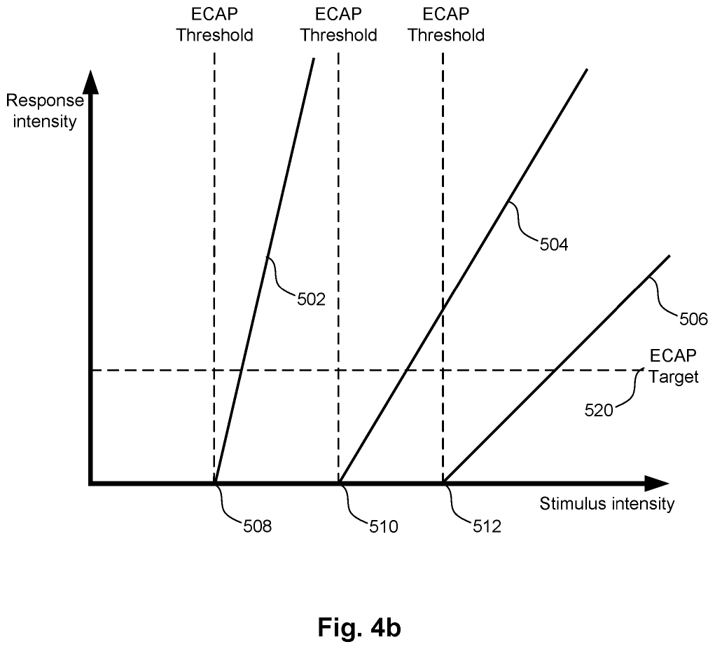

An activation plot, or growth curve, is an approximation to the relationship between stimulus intensity (e.g. an amplitude of the current pulse 160) and intensity of neural response 170 evoked by the stimulus (e.g. an ECAP amplitude). FIG. 4a illustrates an idealised activation plot 402 for one posture of the patient 108. The activation plot 402 shows a linearly increasing ECAP amplitude for stimulus intensity values above a threshold 404 referred to as the ECAP threshold. The ECAP threshold exists because of the binary nature of fibre recruitment; if the field strength is too low, no fibres will be recruited. However, once the field strength exceeds a threshold, fibres begin to be recruited, and their individual evoked action potentials are independent of the strength of the field. The ECAP threshold 404 therefore reflects the field strength at which significant numbers of fibres begin to be recruited, and the increase in response intensity with stimulus intensity above the ECAP threshold reflects increasing numbers of fibres being recruited. Below the ECAP threshold 404, the ECAP amplitude may be taken to be zero. Above the ECAP threshold 404, the activation plot 402 has a positive, approximately constant slope indicating a linear relationship between stimulus intensity and the ECAP amplitude. Such a relationship may be modelled as:

$$y = \{ \begin{matrix}S ( s - T ) , & s \geq T \\ 0 , & s < T\end{matrix} \qquad ( 1 )$$

where s is the stimulus intensity, y is the ECAP amplitude, T is the ECAP threshold and S is the slope of the activation plot (referred to herein as the patient sensitivity). The slope S and the ECAP threshold Tare the key parameters of the activation plot 402.

FIG. 4a also illustrates a discomfort threshold 408, which is a stimulus intensity above which the patient 108 experiences uncomfortable or painful stimulation. FIG. 4a also illustrates a perception threshold 410. The perception threshold 410 corresponds to an ECAP amplitude that is perceivable by the patient. There are a number of factors which can influence the position of the perception threshold 410, including the posture of the patient. Perception threshold 410 may correspond to a stimulus intensity that is greater than the ECAP threshold 404, as illustrated in FIG. 4a, if patient 108 does not perceive low levels of neural activation. Conversely, the perception threshold 410 may correspond to a stimulus intensity that is less than the ECAP threshold 404, if the patient has a high perception sensitivity to lower levels of neural activation than can be detected in an ECAP, or if the signal to noise ratio of the ECAP is low.

For effective and comfortable operation of an implantable neuromodulation device such as the stimulator 100, it is desirable to maintain stimulus intensity within a therapeutic range. A stimulus intensity within a therapeutic range 412 is above the ECAP threshold 404 and below the discomfort threshold 408. In principle, it would be straightforward to measure these limits and ensure that stimulus intensity, which may be closely controlled, always falls within the therapeutic range 412. However, the activation plot, and therefore the therapeutic range 412, varies with the posture of the patient 108.

FIG. 4b illustrates the variation in the activation plots with changing posture of the patient. A change in posture of the patient may cause a change in impedance of the electrode-tissue interface or a change in the distance between electrodes and the neurons. While the activation plots for only three postures, 502, 504 and 506, are shown in FIG. 4b, the activation plot for any given posture can lie between or outside the activation plots shown, on a continuously varying basis depending on posture. Consequently, as the patient's posture changes, the ECAP threshold changes, as indicated by the ECAP thresholds 508, 510, and 512 for the respective activation plots 502, 504, and 506. Additionally, as the patient's posture changes, the slope of the activation plot also changes, as indicated by the varying slopes of activation plots 502, 504, and 506. In general, as the distance between the stimulus electrodes and the spinal cord increases, the ECAP threshold increases and the slope of the activation plot decreases. The activation plots 502, 504, and 506 therefore correspond to increasing distance between stimulus electrodes and spinal cord, and decreasing patient sensitivity.

To keep the applied stimulus intensity within the therapeutic range as patient posture varies, in some implementations an implantable neuromodulation device such as the stimulator 100 may adjust the applied stimulus intensity parameter based on a feedback variable that is determined from one or more measured ECAP characteristics. In one implementation, the device may adjust the stimulus intensity parameter to maintain the feedback loop variable, such as the measured ECAP amplitude at a target response intensity. For example, the device may calculate an error between a target ECAP amplitude and a measured ECAP amplitude, and adjust the applied stimulus intensity parameter to reduce the error as much as possible, such as by adding the scaled error to the current stimulus intensity parameter. A neuromodulation device that operates by adjusting the applied stimulus intensity parameter based on a measured ECAP characteristic is said to be operating in closed-loop mode and will also be referred to as a closed-loop neural stimulation (CLNS) device. By adjusting the applied stimulus intensity parameter to maintain the measured ECAP amplitude at an appropriate target response intensity value, such as a target ECAP amplitude 520 illustrated in FIG. 4b, a CLNS device will generally keep the stimulus intensity within the therapeutic range as patient posture vanes.

A CLNS device comprises a stimulator that takes a stimulus intensity value and converts it into a neural stimulus comprising a sequence of electrical pulses according to a predefined stimulation pattern. The stimulation pattern is parametrised by multiple parameters including stimulus amplitude, pulse width, number of phases, order of phases, number of stimulus electrode poles (two for bipolar, three for tripolar etc.), and stimulus rate or frequency. At least one of the stimulus parameters, for example the stimulus amplitude, is controlled by the feedback loop.

In an example CLNS system, a user (e.g. the patient or a clinician) sets a target response intensity, and the CLNS device performs proportional-integral-differential (PID) control. In some implementations, the differential contribution is disregarded and the CLNS device uses a first order integrating feedback loop. The stimulator produces stimulus in accordance with a stimulus intensity parameter, which evokes a neural response in the patient. The intensity of an evoked neural response (e.g. an ECAP) is measured by the CLNS device and compared to the target response intensity.

The measured neural response intensity, and its deviation from the target response intensity, is used by the feedback loop to determine possible adjustments to the stimulus intensity parameter to maintain the neural response at the target intensity. If the target intensity is properly chosen, the patient receives consistently comfortable and therapeutic stimulation through posture changes and other perturbations to the stimulus/response behaviour.

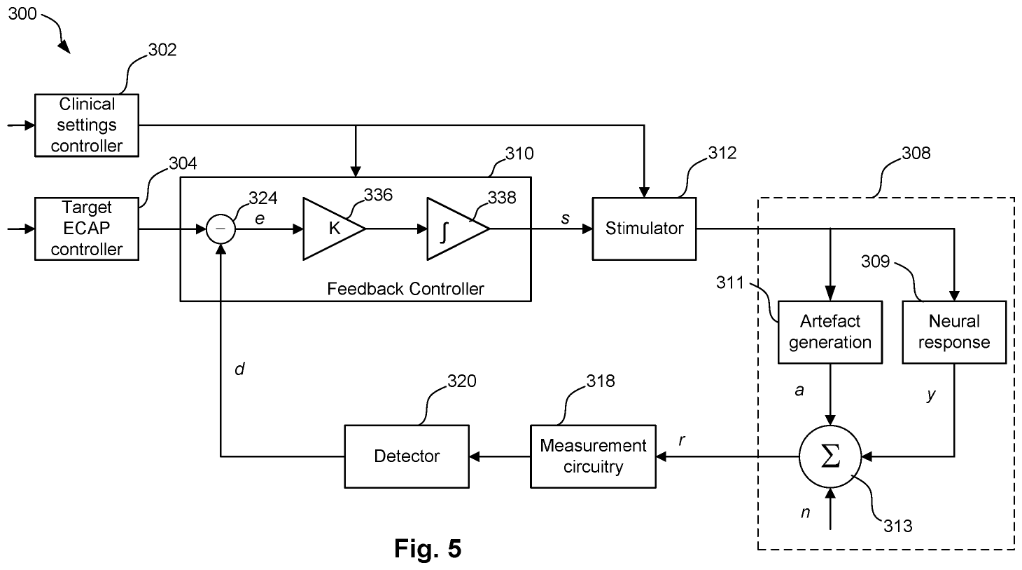

FIG. 5 is a schematic illustrating elements and inputs of a closed-loop neural stimulation system (CLNS) 300, according to one implementation of the present technology. The system 300 comprises a stimulator 312 which converts a stimulus intensity parameter (for example a stimulus current amplitude) s, in accordance with a set of predefined stimulus parameters, to a neural stimulus comprising a sequence of electrical pulses on the stimulus electrodes (not shown in FIG. 5). According to one implementation, the predefined stimulus parameters comprise the number and order of phases, the number of stimulus electrode poles, the pulse width, and the stimulus rate or frequency.

The generated stimulus crosses from the electrodes to the spinal cord, which is represented in FIG. 5 by the dashed box 308. The box 309 represents the evocation of a neural response y by the stimulus as described above. The box 311 represents the evocation of an artefact signal a, which is dependent on stimulus intensity and other stimulus parameters, as well as the electrical environment of the measurement electrodes. Various sources of measurement noise n, as well as the artefact a, may add to the evoked response y at the summing element 313 to form the sensed signal r, including: electrical noise from external sources such as 50 Hz mains power; electrical disturbances produced by the body such as neural responses evoked not by the device but by other causes such as peripheral sensory input; EEG; EMG; and electrical noise from measurement circuitry 318.

The neural recruitment arising from the stimulus is affected by mechanical changes, including posture changes, walking, breathing, heartbeat and so on. Mechanical changes may cause impedance changes, or changes in the location and orientation of the nerve fibres relative to the electrode array(s). As described above, the intensity of the evoked response provides a measure of the recruitment of the fibres being stimulated. In general, the more intense the stimulus, the more recruitment and the more intense the evoked response. An evoked response typically has a maximum amplitude in the range of microvolts, whereas the voltage resulting from the stimulus applied to evoke the response is typically several volts.

Measurement circuitry 318, which may be identified with measurement circuitry 128, amplifies the sensed signal r (including evoked neural response, artefact, and measurement noise) and samples the amplified sensed signal r to capture a series of “signal windows” each comprising a predetermined number of samples of the amplified sensed signal r. The ECAP detector 320 processes the signal window and outputs a measured neural response intensity d. A typical number of samples in a captured signal window is 60. In one implementation, the neural response intensity comprises a peak-to-peak ECAP amplitude. The measured response intensity d is input into the feedback controller 310. The feedback controller 310 comprises a comparator 324 that compares the measured response intensity d (an example of a feedback variable) to a target ECAP amplitude as set by the target ECAP controller 304 and provides an indication of the difference between the measured response intensity d and the target ECAP amplitude. This difference is the error value, e.

The feedback controller 310 calculates an adjusted stimulus intensity parameter, s, with the aim of maintaining a measured response intensity d equal to the target ECAP amplitude. Accordingly, the feedback controller 310 adjusts the stimulus intensity parameter s to minimise the error value, e. In one implementation, the controller 310 utilises a first order integrating function, using a gain element 336 and an integrator 338, in order to provide suitable adjustment to the stimulus intensity parameter s. According to such an implementation, the current stimulus intensity parameter s may be determined by the feedback controller 310 as

$$s = \int K e d t \qquad ( 2 )$$

where K is the gain of the gain element 336 (the controller gain). This relation may also be represented as

$$δ s = K e \qquad ( 3 )$$

where δs is an adjustment to the current stimulus intensity parameter s.

A target ECAP amplitude is input to the feedback controller 310 via the target ECAP controller 304. In one embodiment, the target ECAP controller 304 provides an indication of a specific target ECAP amplitude. In another embodiment, the target ECAP controller 304 provides an indication to increase or to decrease the present target ECAP amplitude. The target ECAP controller 304 may comprise an input into the CLNS system 300, via which the patient or clinician can input a target ECAP amplitude, or the indication thereof. The target ECAP controller 304 may comprise memory in which the target ECAP amplitude is stored, and from which the target ECAP amplitude is provided to the feedback controller 310.

A clinical settings controller 302 provides clinical settings to the system 300, including the feedback controller 310 and the stimulus parameters for the stimulator 312 that are not under the control of the feedback controller 310. In one example, The clinical settings controller 302 may be configured to adjust the controller gain K of the feedback controller 310 to adapt the feedback loop to patient sensitivity. The clinical settings controller 302 may comprise an input into the neuromodulation device, via which the patient or clinician can adjust the clinical settings. The clinical settings controller 302 may comprise memory in which the clinical settings are stored, and are provided to components of the system 300.

In some implementations, two clocks (not shown) are used, being a stimulus clock operating at the stimulus frequency (e.g. 60 Hz) and a sample clock for sampling the sensed signal r (for example, operating at a sampling frequency of 10 kHz). As the ECAP detector 320 is linear, only the stimulus clock affects the dynamics of the CLNS system 300. On the next stimulus clock cycle, the stimulator 312 outputs a stimulus in accordance with the adjusted stimulus intensity s. Accordingly, there is a delay of one stimulus clock cycle before the stimulus intensity is updated in light of the error value e.

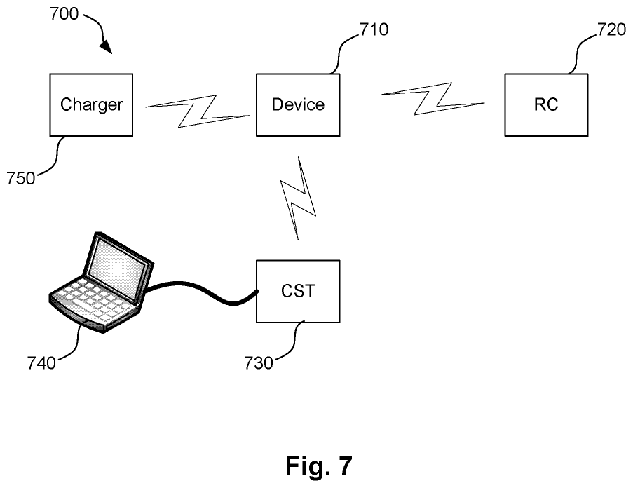

FIG. 7 is a block diagram of a neural stimulation system 700. The neural stimulation system 700 is centred on a neuromodulation device 710. In one example, the neuromodulation device 710 may be implemented as the stimulator 100 of FIG. 1, implanted within a patient (not shown). The neuromodulation device 710 is connected wirelessly to a remote controller (RC) 720. The remote controller 720 is a portable computing device that provides the patient with control of their stimulation in the home environment by allowing control of the functionality of the neuromodulation device 710, including one or more of the following functions: enabling or disabling stimulation; adjustment of stimulus intensity or target neural response intensity; and selection of a stimulation control program from the control programs stored on the neuromodulation device 710.

The charger 750 is configured to recharge a rechargeable power source of the neuromodulation device 710. The recharging is illustrated as wireless in FIG. 7 but may be wired in alternative implementations.

The neuromodulation device 710 is wirelessly connected to a Clinical System Transceiver (CST) 730. The wireless connection may be implemented as the transcutaneous communications channel 190 of FIG. 1. The CST 730 acts as an intermediary between the neuromodulation device 710 and the Clinical Interface (CI) 740, to which the CST 730 is connected. A wired connection is shown in FIG. 7, but in other implementations, the connection between the CST 730 and the CI 740 is wireless.

The CI 740 may be implemented as the external computing device 192 of FIG. 1. The CI 740 is configured to program the neuromodulation device 710 and recover data stored on the neuromodulation device 710. This configuration is achieved by program instructions collectively referred to as the Clinical Programming Application (CPA) and stored in an instruction memory of the CI 740.

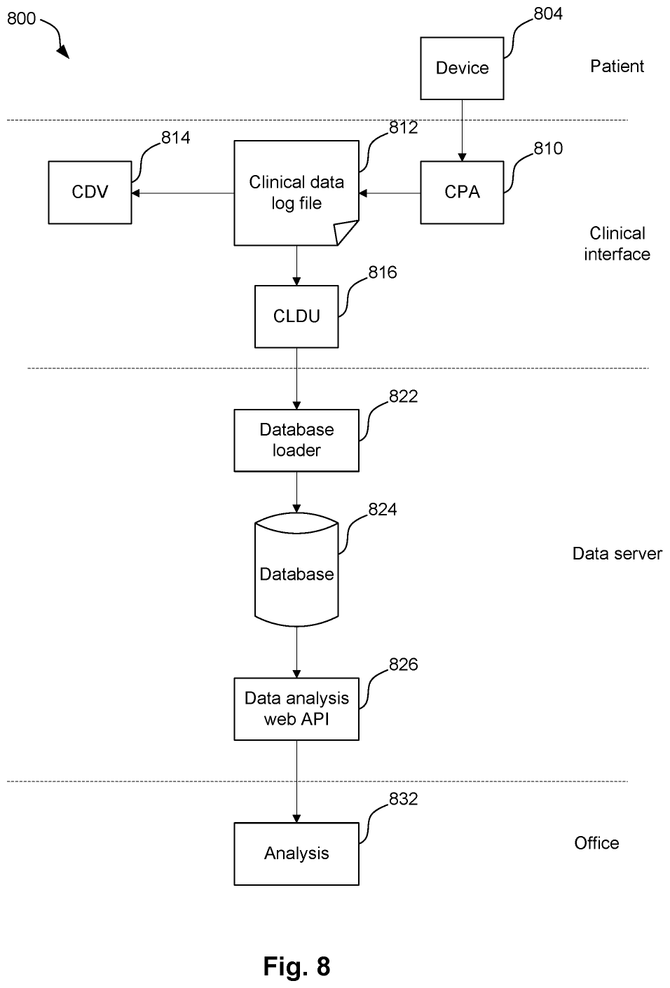

FIG. 8 is a block diagram illustrating the data flow 800 of a neural stimulation therapy system such as the system 700 of FIG. 7 according to one implementation of the present technology. Neuromodulation device 804, once implanted within a patient, applies stimuli over a potentially long period such as weeks or months and records neural responses, clinical settings, paraesthesia target level, and other operational parameters, discussed further below. Neuromodulation device 804 may comprise a Closed-Loop Neural Stimulation (CLNS) device, in that the recorded neural responses are used in a feedback arrangement to control clinical settings on a continuous or ongoing basis. To effect suitable SCS therapy, neuromodulation device 804 may deliver tens, hundreds or even thousands of stimuli per second, for many hours each day. The feedback loop may operate for most or all of this time, by obtaining sensed signals subsequent to every stimulus, or at least obtaining such sensed signals regularly. Each sensed signal generates a feedback variable such as a measure of the amplitude of the evoked neural response, which in turn results in the feedback loop changing at least one stimulus parameter for a following stimulus. Neuromodulation device 804 thus produces such data at a rate of tens or hundreds of Hz, or even kHz, and over the course of hours or days this process results in large amounts of clinical data. This is unlike past neuromodulation devices such as open-loop SCS devices which lack any ability to record any neural response.

When brought in range with a receiver, neuromodulation device 804 transmits data, e.g. via telemetry module 114, to a clinical programming application (CPA) 810 installed on a clinical interface. In one implementation, the clinical interface is the CI 740 of FIG. 7. The data can be grouped into two main sources: (1) Data collected in real-time during a programming session; (2) Data downloaded from a stimulator after a period of non-clinical use by a patient. CPA 810 collects and compiles the data into a clinical data log file 812.

All clinical data transmitted by the neuromodulation device 804 may be compressed by use of a suitable data compression technique before transmission by telemetry module 114 and/or before storage into the memory 118 to enable storage by neuromodulation device 804 of higher resolution data. This higher resolution allows neuromodulation device 804 to provide more data for post-analysis and more detailed data mining for events during use. Alternatively, compression enables faster transmission of standard-resolution clinical data.

The clinical data log file 812 is manipulated, analysed, and efficiently presented by a clinical data viewer (CDV) 814 for field diagnosis by a clinician, field clinical engineer (FCE) or the like. CDV 814 is a software application installed on the Clinical Interface (CI). In one implementation, CDV 814 opens one Clinical Data Log file 812 at a time. CDV 814 is intended to be used in the field to diagnose patient issues and optimise therapy for the patient. CDV 814 may be configured to provide the user or clinician with a summary of neuromodulation device usage, therapy output, and errors, in a simple single-view page immediately after log files are compiled upon device connection.

Clinical Data Uploader 816 is an application that runs in the background on the CI, that uploads files generated by the CPA 810, such as the clinical data log file 812, to a data server. Database Loader 822 is a service which runs on the data server and monitors the patient data folder for new files. When Clinical Data Log files are uploaded by Clinical Data Uploader 816, database loader 822 extracts the data from the file and loads the extracted data to Database 824.

The data server further contains a data analysis web API 826 which provides data for third-party analysis such as by the analysis module 832, located remotely from the data server. The ability to obtain, store, download and analyse large amounts of neuromodulation data means that the present technology can: improve patient outcomes in difficult conditions; enable faster, more cost effective and more accurate troubleshooting and patient status; and enable the gathering of statistics across patient populations for later analysis, with a view to diagnosing aetiologies and predicting patient outcomes.

### The Assisted Programming System

As mentioned above, obtaining patient feedback about their sensations is important during programming of closed-loop neural stimulation therapy, but mediation by trained clinical engineers is expensive and time-consuming. It would therefore be advantageous if patients could program their own implantable device themselves, or with some assistance from a clinician. However, interfaces for current programming systems are non-intuitive and generally unsuitable for direct use by patients because of their technical nature. There is therefore a need for a CPA to be as intuitive for non-technical users as possible while avoiding discomfort to the patient.

Implementations of an Assisted Programming System (APS) according to the present technology are generally configured to meet this need.

In some implementations, the APS comprises two elements: the Assisted Programming Module (APM), which forms part of the CPA, and the Assisted Programming Firmware (APF), which forms part of the control programs 122 executed by the controller 116 of the electronics module 110. The data obtained from the patient is analysed by the APM to determine the parameters and settings for the neural stimulation therapy to be delivered by the stimulator 100. The APF is configured to complement the operation of the APM by responding to commands issued by the APA via the CST 730 to the stimulator 100 to deliver specified stimuli to the patient, and by returning, via the CST 730, measurements of neural responses to the delivered stimuli.

In other implementations, all the processing of the APS according to the present technology is done by the APF. In other words, the data obtained from the patient is not passed to the APM, but is analysed by the APF to determine the parameters and settings for the neural stimulation therapy to be delivered by the stimulator 100.

In implementations of the APS in which the APM analyses the data from the patient, the APS instructs the device 710 to capture and return signal windows to the CI 740 via the CST 730. In such implementations, the device 710 captures the signal windows using the measurement circuit 128 and bypasses the ECAP detector 320, storing the data representing the raw signal windows temporarily in memory 118 before transmitting the data representing the captured signal windows to the APS for analysis.

Following the processing, the APS may load the determined program onto the device 710 to govern subsequent neural stimulation therapy. In one implementation, the program comprises clinical settings 121 that are input to the neuromodulation device by or stored in, the clinical settings controller 302. The patient may subsequently control the device 710 to deliver the therapy according to the determined program using the remote controller 720 as described above. The determined program may also, or alternatively, be loaded into the CPA for validation and modification.

### Loop Stability Measures

The operation of the closed-loop neural stimulation system is governed by loop variables that include, but are not limited to, stimulus intensity, an error value, and the characteristics of the ECAP. The stimulus intensity may include a current value or a voltage value of the stimulus pulses that are applied to the tissue. In the closed-loop mode, the stimulus intensity is modified based on the other loop variables, making the stimulus intensity loop variable representative of loop stability or, more generally, loop operation. Further, the error value is the difference between the feedback variable and a target value, as explained in conjunction with FIG. 5. It is important to note that the error value can be derived by using any characteristic associated with the ECAP. For example, the feedback variable could be the amplitude of the ECAP. Consequently, the error value can be the difference between the measured amplitude of the ECAP and the target ECAP amplitude that was set during the fitting process. A person skilled in the art would appreciate that the error value depends on the feedback variable selected to control the loop. Further, the feedback variable is another parameter that governs the operation of the loop in a closed-loop SCS system. The feedback variable could be any characteristic of the neural response such as an ECAP. The characteristic of the neural response could be, for example, but not limited to, a peak-to-peak amplitude, the frequency, or a spectral characteristic of the neural response.

### Loop Stability Measures—Frequency Domain Approach

One approach to determining loop stability in a CLNS system may be using a frequency domain analysis of the loop variables. For instance, the frequency content of the loop variables that include, but are not limited to, stimulus current, the feedback variable and the error value may be analysed to detect an anomalous condition of loop performance. Digital signal processing techniques may be used to derive the frequency content of the loop variables. The anomalous condition may be, for example, peaks of a certain height above the background at particular frequencies in a Fourier (frequency) spectrum. Further, spectral characteristics of the loop response may be observed to detect anomalies. In some instances, perturbations may be introduced during the loop operation to detect the instability in its operation.

Anomalous conditions in loop performance may manifest in various ways. In an instance, it is a sign of instability if the sign of the error value changes with every stimulus, which may be detected as a significant component of oscillation at half the stimulus or Nyquist frequency. As a precursor one may exclude likely imposed sources of oscillation such as heartbeat (around 1-3 Hz) before analysing the loop stability in this manner. The oscillation sources such as heartbeat and breathing may be rejected by selecting an appropriate corner/cut-off frequency.

### Loop Stability Measures—Time-Domain Approach

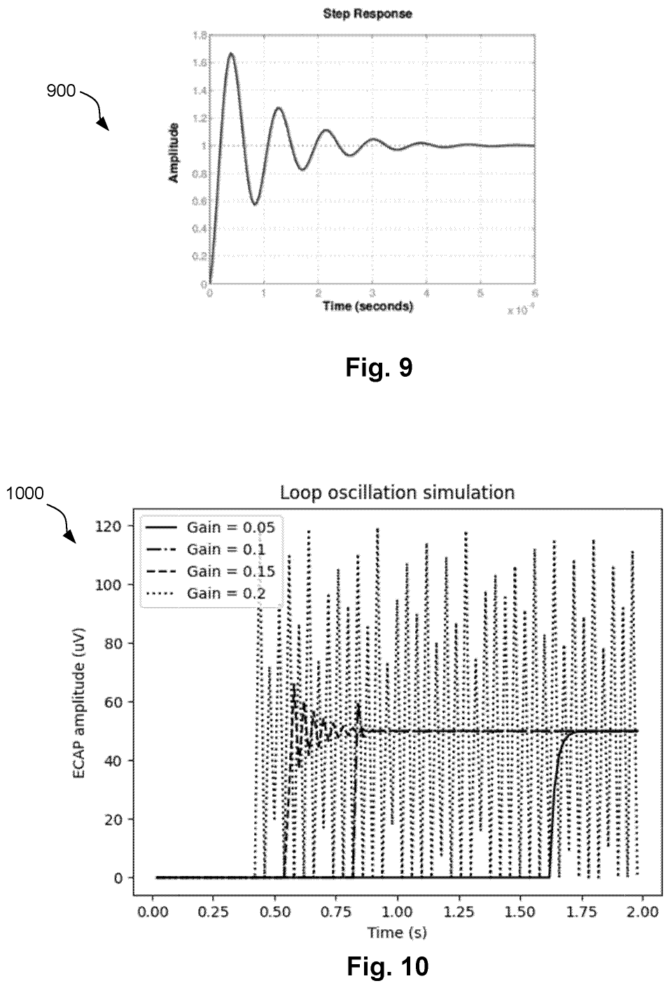

In an implementation, time-domain features of the feedback loop variables are used to determine the stability measure of the loop. Time-domain features may include step response, impulse response, or other transient response. In addition to the loop response, histograms that store the loop variables may be used to determine a measure of loop performance. In an instance, instability in the closed-loop operation may be detected by applying a step change to the loop. FIG. 9 illustrates a generic under-damped step response 900 of a closed-loop control system. A step-change could be introduced by changing the target value, which could be a target neural response intensity, or the stimulation current. The response of the loop may be observed upon introducing the step change. If a loop variable exhibits the characteristics of an under-damped response, as illustrated in FIG. 9, it indicates proximity to the threshold conditions for loop instability. The extent of the under-damping may be quantified and displayed as a loop stability measure.

FIG. 10 illustrates exemplary loop oscillation time series for different controller gain values on a fixed-size step change to the target value. Chart 1000 shows the ECAP amplitude as the loop variable on the y-axis and time in seconds on the x-axis. In chart 1000 we observe that the loop oscillation varies for different gain values. In an instance, for a gain value of 0.05, the control loop latches onto the target value after some delay without any overshoot. However, at a gain value of 0.1, we can observe that the loop is taking a lesser time to reach the target value but there is a slight overshoot before settling. Further, at a gain value of 0.15, the loop responds faster toward reaching the target value but oscillates for 0.25 seconds before achieving a steady state. Furthermore, at a gain value of 0.2, the loop variable oscillates continuously and is said to be unstable. The oscillation is at half the stimulus rate.

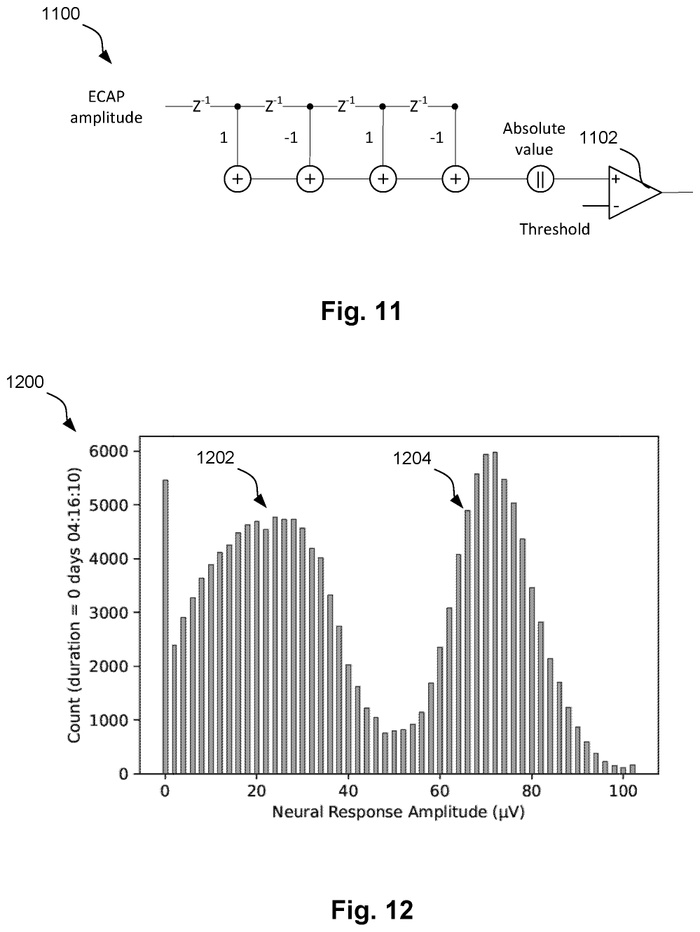

FIG. 11 illustrates a filter 1100 that is configured to detect loops that are unstable, according to an implementation. Filter 1100 of FIG. 11 is a Finite Impulse Response (FIR) filter that detects loops that are unstable, such as the loop with a gain of 0.2 shown in FIG. 10. The filter 1100 is configured to receive, for example, a loop variable such as amplitudes of neural response or error values, and detect instability of the loop. For example, the filter 1100 may be a four-sample FIR filter that is sensitive to alternating signals, i.e., if the inputs have opposite signs with each sample (i.e. oscillation at half the stimulus rate), they accumulate. Thereafter, the absolute value of the sum of the alternating signals is computed and compared to a threshold value. If the absolute value exceeds a threshold, then the loop conditions are deemed unstable. A comparator 1102 may be used to compare the absolute value of the sum of the alternating signals with the threshold value. The number of samples in the FIR filter and the threshold may be adjusted based on the desired performance.

Another example of detecting instability in the time domain is a looking for a bimodal histogram of loop variables such as, but not being limited to, stimulus intensity or feedback variable. FIG. 12 illustrates a histogram 1200 with a bimodal distribution of neural response amplitude indicating an unstable loop. For example, if stimulus intensity or neural response intensity oscillates between two values, it leads to a bimodal distribution in the histogram comprising two discrete distribution peaks 1202 and 1204. A statistical measure of bimodality may be used to quantify this measure of instability. For example, the statistical measure could be the presence of more than one normal distribution in the generated histogram. One such measure is Hartigan's dip test.

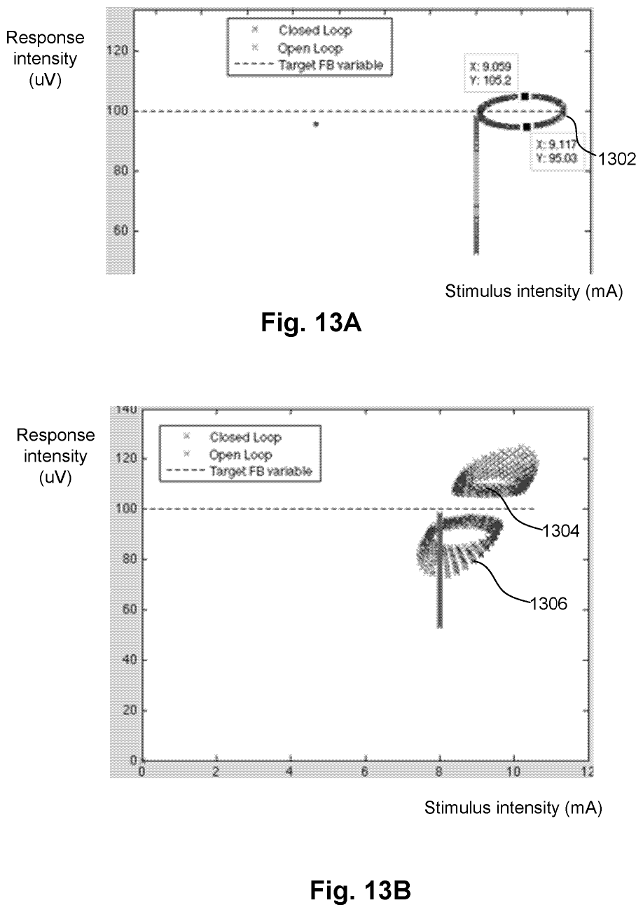

FIGS. 13A and 13B illustrate open loop and closed loop performance at various frequencies and gain values using Lissajous figures, according to an implementation. Lissajous figures plot the feedback variable (neural response intensity) against the control variable (stimulus intensity). In the present technology, we only consider the performance of the closed-loop system and ignore the open-loop performance. The ellipse 1302 in the Lissajous figure of FIG. 13A illustrates stable performance of the closed-loop controller at a particular gain value and stimulus frequency. However, in FIG. 13B, the shape 1304 has a reflection shape 1306 in the lower half and is not following an elliptical path. This bi-lobular behaviour indicates that the closed-loop performance is unstable. The processor associated with the implantable device may determine a stability measure based on the Lissajous figure, for example as a measure of bi-lobularity of the Lissajous figure.

Another example of detecting instability in the time domain relies on the relationship between noise in the stimulus intensity parameter s and the loop gain G (the product of the controller gain K and the patient sensitivity S). It may be shown that the stimulus intensity noise ratio Rₛ, defined as the standard deviation σₛ of the noise in the stimulus intensity parameter s as a proportion of the therapeutic range Δs, is given by the following equation:

$${R}_{s} = \frac{{σ}_{s}}{Δ s} = \frac{1}{2 \cdot SNR} \sqrt{\frac{G}{2 - G}} \qquad ( 4 )$$

where SNR is the measurement signal to noise ratio. The stimulus intensity noise ratio Rₛ may be measured by estimating the standard deviation σₛ of the noise in the stimulus intensity parameter s and dividing by the therapeutic range Δs.

The stimulus intensity noise ratio Rₛ increases from zero at G=0 (open-loop) to infinity (complete instability) as loop gain G approaches 2. The loop enters the under-damped region when G=1, at which value the stimulus intensity noise ratio Rₛ is equal to 1/(2*SNR). A loop stability metric may therefore be determined from the stimulus intensity s (the loop variable in this example) as the ratio rₛ of the measured stimulus intensity noise ratio Rₛ to its value at G=1, namely 1/(2SNR). The ratio rₛ is less than 1 for a stable loop, equal to 1 at G=1, and exceeds 1 for an under-damped loop.

Another example of detecting instability in the time domain relies on the relationship between noise in the measured response intensity d and the loop gain G. It may be shown that the response intensity noise ratio R_{d}, defined as the standard deviation σ_{d} of the measurement noise in the response intensity d in closed-loop mode to the value of σ_{d} in open-loop mode (i.e. G=0), is given by the following equation:

$${R}_{d} = \frac{{σ}_{d} ( closed )}{{σ}_{d} ( G = 0 )} = \sqrt{\frac{2}{2 - G}} \qquad ( 5 )$$

The response intensity noise ratio R_{d} may be measured by estimating the standard deviation σ_{d} of the noise in the response intensity d and dividing by the open-loop value of σ_{d}, which may be measured by setting the controller gain K to zero.

The response intensity noise ratio R_{d} increases from one at G=0 (open-loop) to infinity (complete instability) as loop gain G approaches 2. The loop enters the under-damped region when G=1, at which value the response intensity noise ratio R_{d} is equal to 1.414. A loop stability metric may therefore be determined as the response intensity noise ratio R_{d}, whose value is less than 1.414 for a stable loop, equal to 1.414 at G=1, and exceeds 1.414 for an under-damped loop.

In cases where the stability measure indicates instability, the program parameters governing the closed loop operation may be adjusted and/or actions to mitigate the instability may be taken based on the loop stability measure.

### Actions Based on the Loop Stability Measure

An implantable neuromodulation device such as the device 100 may be configured to take appropriate actions based on the determined loop stability measure. The actions may be taken with a view to improve the loop stability and prevent potential malfunction of the implantable device. The actions may include, but are not limited to, adjusting the stimulus intensity, changing the measurement electrodes, adjusting a target value, adjusting the controller gain value, and transitioning to an open-loop mode. In some cases, the action may include transmitting an error code to an external device based on the loop stability measure. For example, the implantable device may transmit an interrupt signal to an external device that generates and/or issues an alert to indicate to the user that there is an issue with the implantable device. Further, the user may take remedial action or consult a physician for assistance.

In some implementations, the programming system may be configured to take appropriate actions to restore loop stability. For example, the Assisted Programming System (APS) may include a loop stability module that analyses the loop variables during programming to ensure a stable operation. The loop stability module may be configured to assess time-domain and/or frequency-domain aspects of the loop variables and determine a loop stability measure. Further, the loop stability module may take appropriate actions, based on the loop stability measure, to restore loop stability. The loop stability module may take actions that include, but are not limited to, adjusting the program parameters, broadcasting an alert to the user, transitioning to an open-loop operation, and shutting down the implantable device.

Actions such as changing stimulus intensity and adjusting the target value may be useful in tuning the loop performance based on the program parameters used to set up the loop. Further, actions such as changing a controller gain value will affect the way the loop responds to changes in the loop variables. The controller gain may be changed within an upper bound and a lower bound and may not be allowed to be set to any random value. Further, the controller gain value may not be accessible to the user and may require a clinician to adjust the controller gain value.

In some cases, when the closed-loop performance becomes unstable, as in the example in FIG. 10, the processor or the control unit of the implantable device may transition the loop from a closed-loop mode to an open-loop mode. In this manner, the user or the patient continues to receive therapy while the loop will not display anomalous behaviour.

### Methods of Determining Loop Stability Measures

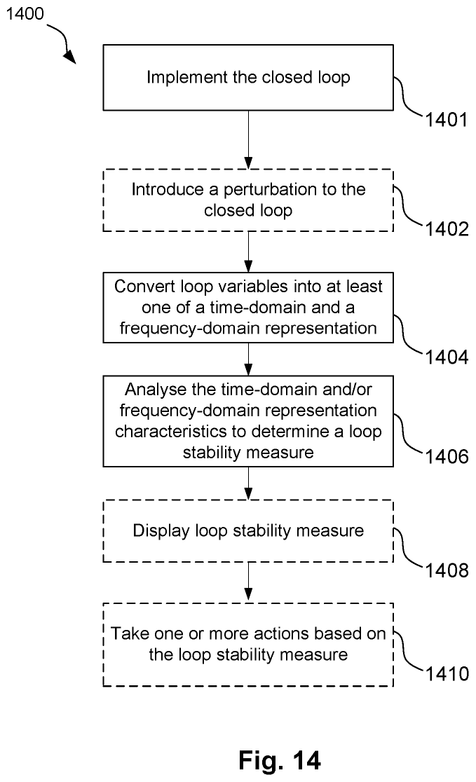

FIG. 14 illustrates a flow chart 1400 pertaining to the method steps for assessing the stability of a loop, in accordance with an implementation. Generally, the method steps are executed by the control unit, which is a part of a computing device such as controller 116, of the implantable device or an external device like the clinical interface 740. In step 1401, the closed-loop neural stimulation system is implemented. In optional step 1402, a perturbation may be introduced in the closed-loop system to examine the transient response. The perturbation may be in the form of changing a target value or changing the stimulation intensity. Further, in step 1404, the loop variables are converted into at least one of a time-domain representation and a frequency-domain representation. The loop variables include, but are not limited to, error value, wherein the error value is the difference between the feedback variable and a target value, the feedback variable, and a stimulus intensity parameter. The time-domain representation may include but is not limited to, time series, Lissajous figures, and a histogram. Further, the frequency-domain representation may include, but is not limited to, a Fourier spectrum. In step 1406, the characteristics of the time-domain and/or the frequency-domain representation are analysed and one or more loop stability measures are determined. The loop stability measure may indicate the stability of the closed loop operation. At optional step 1408, the loop stability measure is displayed. The loop stability measure may be displayed on the screen of one or more external devices in communication with the implantable device, such as the remote control (RC) 720 and the clinical interface 740. In some embodiments, the clinical programming application (CPA) may determine the loop stability measure and display the same on the screen of the CI 740. Further, in optional step 1410, one or more actions that tune the loop operation may be taken based on the loop stability measure. The actions may be at least one of, but not limited to, adjusting at least one program parameter, transitioning to open-loop operation, issuing an alert to one of the external devices, or shutting down the implantable device.

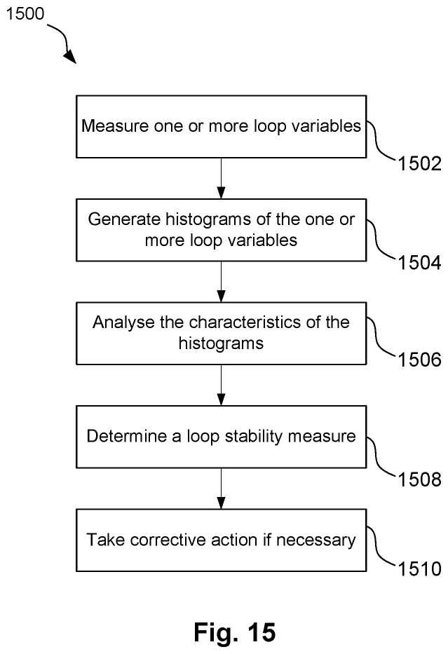

FIG. 15 illustrates a flowchart pertaining to the steps of a method 1500 of using a histogram for assessing the stability of the loop operation. Generally, the method steps are executed by the control unit, which is a part of a computing device such as controller 116, of the implantable device or an external device like the clinical interface 740. In some instances, the histogram may be at least one of a one-dimensional histogram and a two-dimensional histogram. At step 1502, one or more loop variables are monitored and/or measured continuously. At step 1504, histograms of the loop variables are generated. At step 1506, the characteristics of the histograms are analysed by the control unit. The characteristics may include, but are not limited to, a statistical measure such as bimodality of the distribution of values in the histograms, and other measures. At step 1508, a loop stability measure is determined based on the analysis of the histograms. At optional step 1510, one or more corrective actions may be taken by the control unit to stabilise the loop operation, if necessary.

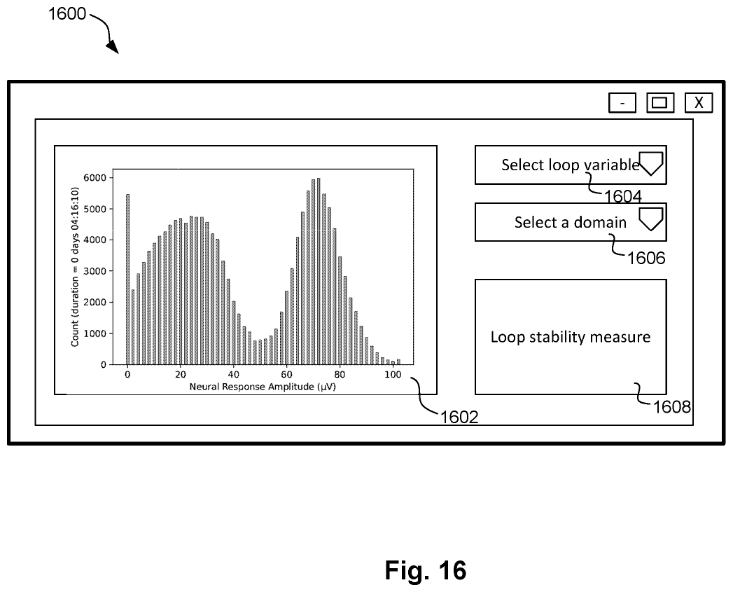

FIG. 16 illustrates a user interface 1600 for assessing the loop variables and displaying a loop stability measure. The user interface 1600 may be rendered on any external device such as remote control 720, a clinical interface 740 or any other device such as a smartphone or a computing device in communication with the implantable neuromodulation device. The user interface 1600 may include a drop-down menu 1604 that enables a user to select at least one loop variable of interest. Further, the user interface 1600 may include another drop-down menu 1606 that enables the user to select the domain in which the user would like to view the loop stability characteristics. For example, menu 1606 may include the options for time-domain analysis or frequency-domain analysis. The user interface 1600 further may include a display area 1602, where a loop variable representation is displayed based on the selections of the user from menus 1604 and 1606. In the example of FIG. 16, the selected loop variable from menu 1604 was neural response amplitude, while the selected domain of analysis from menu 1606 was the time domain, so the displayed representation in the display area 1602 is a histogram of the neural response amplitude. Further, in display area 1608, the loop stability measure determined from the selected loop variable in the selected domain is displayed.

It will be appreciated by persons skilled in the art that numerous variations and/or modifications may be made to the invention as shown in the specific implementations without departing from the spirit or scope of the invention as broadly described. The present implementations are, therefore, to be considered in all respects as illustrative and not limiting or restrictive.

LABEL LIST

| | |
|---|---|
| stimulus electrode | 2 |
| return electrode | 4 |
| measurement electrodes | 6 |
| reference electrode | 8 |
| implanted stimulator | 100 |
| patient | 108 |
| electronics module | 110 |
| battery | 112 |
| telemetry module | 114 |
| controller | 116 |
| memory | 118 |
| remote control RC | 120 |
| clinical settings | 121 |
| control programs | 122 |
| pulse generator | 124 |
| electrode selection module | 126 |
| measurement circuitry | 128 |
| system ground | 130 |
| electrode array | 150 |
| biphasic stimulus pulse | 160 |
| neural response | 170 |
| nerve | 180 |
| transcutaneous communications channel | 190 |
| external device | 192 |
| closed - loop neural stimulation system | 300 |
| clinical settings controller | 302 |
| target ECAP controller | 304 |
| box | 308 |
| box | 309 |
| feedback controller | 310 |
| box | 311 |
| stimulator | 312 |
| measurement circuitry | 318 |
| ECAP detector | 320 |
| comparator | 324 |
| gain element | 336 |
| integrator | 338 |
| activation plot | 402 |
| ECAP threshold | 404 |
| discomfort threshold | 408 |
| perception threshold | 410 |
| therapeutic range | 412 |
| activation plot | 502 |
| activation plot | 504 |
| activation plot | 506 |
| ECAP threshold | 508 |
| ECAP threshold | 510 |
| ECAP threshold | 512 |
| ECAP target | 520 |
| single - ended ECAP | 600 |
| neuromodulation system | 700 |
| neuromodulation device | 710 |
| remote controller | 720 |
| clinical System Transceiver | 730 |
| clinical Interface | 740 |
| charger | 750 |
| generic step response | 900 |
| chart | 1000 |
| filter | 1100 |
| histogram | 1200 |
| peak | 1202 |
| peak | 1204 |
| ellipse | 1302 |
| shape | 1304 |
| shape | 1306 |
| flow chart | 1400 |
| step | 1402 |
| step | 1404 |
| step | 1406 |
| step | 1408 |
| step | 1410 |
| flowchart | 1500 |
| step | 1502 |
| step | 1504 |
| step | 1506 |
| step | 1508 |
| step | 1510 |
| user interface | 1600 |
| display area | 1602 |
| drop - down menu | 1604 |
| drop - down menu | 1606 |
| display area | 1608 |

1. An implantable neuromodulation device comprising:
a plurality of electrodes including one or more stimulus electrodes and one or more measurement electrodes;
a stimulus source configured to provide neural stimuli to be delivered via the one or more stimulus electrodes to a neural pathway of a patient in order to evoke a neural response on the neural pathway;
measurement circuitry configured to capture signal windows sensed on the neural pathway via the one or more measurement electrodes subsequent to respective neural stimuli; and
a control unit configured to:
control the stimulus source to provide a neural stimulus according to a stimulus intensity parameter;
measure an intensity of the evoked neural response in the captured signal window subsequent to the provided neural stimulus;
determine a feedback variable from the measured intensity of the evoked neural response;
implement a feedback loop by using the feedback variable to control the stimulus intensity parameter so as to maintain the feedback variable at a target value;
convert a loop variable of the feedback loop to a representation, wherein the representation is one of a time-domain representation and a frequency-domain representation; and
analyse the representation to determine a loop stability measure of the feedback loop.

2. The device of claim 1, wherein the loop variable comprises one of: the feedback variable; an error value between the feedback variable and the target value; and the stimulus intensity parameter.

3. The device of claim 1, wherein the representation is a time-domain representation.

4. The device of claim 3, wherein the time-domain representation is a histogram.

5. The device of claim 4, wherein the control unit is configured to analyse the representation by determining a measure of bimodality of the histogram as the loop stability measure.

6. The device of claim 3, wherein the time-domain representation is a Lissajous figure.

7. The device of claim 6, wherein the control unit is configured to analyse the representation by determining a measure of bilobularity of the Lissajous figure as the loop stability measure.

8. The device of claim 3, wherein the time-domain representation is a time series.

9. The device of claim 8, wherein the control unit is configured to analyse the representation using a finite impulse response filter to detect an oscillation in the time series at a predetermined frequency.

10. The device of claim 9, wherein the predetermined frequency is half of a frequency of the provided neural stimuli.

11. The device of claim 8, wherein the control unit is further configured to introduce a perturbation to the feedback loop before the converting.

12. The device of claim 11, wherein the perturbation is a step.

13. The device of claim 12, wherein the control unit is configured to analyse the representation by determining a measure of under-dampedness of the time series as the loop stability measure.

14. The device of claim 8, wherein the control unit is configured to analyse the representation by determining a ratio of a standard deviation of stimulus intensity noise to a therapeutic range.

15. The device of claim 8, wherein the control unit is configured to analyse the representation by determining a ratio of a standard deviation of noise of the feedback variable to a standard deviation of noise of the feedback variable in open-loop mode.

16. The device of claim 1, wherein the representation is a frequency domain representation.

17. The device of claim 16, wherein the representation is a Fourier spectrum of the loop variable.

18. The device of claim 17, wherein the control unit is configured to analyse the representation by determining the magnitude of the Fourier spectrum at a predetermined frequency as the loop stability measure.

19. The device of claim 18, wherein the predetermined frequency is half of a frequency of the provided neural stimuli.

20. The device of claim 1, wherein the control unit is further configured to take one or more actions based on the loop stability measure.

21. The device of claim 20, wherein the control unit is further configured to take one or more actions based on a comparison of the loop stability measure and a predetermined threshold.

22. The device of claim 20, wherein the one or more actions comprise one or more of: adjusting one or more program parameters of the feedback loop; issuing an alert; transitioning to an open-loop operation of the implantable neuromodulation device; and shutting down the implantable neuromodulation device.

23. A method of determining a measure of stability of a closed-loop neural stimulation system, the method comprising:
delivering a neural stimulus to a neural pathway of a patient in order to evoke a neural response on the neural pathway, the neural stimulus being delivered according to a stimulus intensity parameter;
capturing a signal window sensed on the neural pathway subsequent to the delivered neural stimulus;
measuring an intensity of the neural response evoked by the delivered neural stimulus in the captured signal window;
determining, from the measured intensity of the evoked neural response, a feedback variable;
implementing a feedback loop by using the determined feedback variable to control the stimulus intensity parameter so as to maintain the feedback variable at a target value;
converting a loop variable of the feedback loop to a representation, wherein the representation is one of a time-domain representation and a frequency-domain representation; and
analysing the representation to determine a loop stability measure of the feedback loop.

24. The method of claim 23, wherein the loop variable comprises one of: the feedback variable; an error value between the feedback variable and the target value; and the stimulus intensity parameter.

25. The method of claim 23, wherein the representation is a time-domain representation.

26. The method of claim 25, wherein the time-domain representation is a histogram.

27. The method of claim 25, wherein the time-domain representation is a Lissajous figure.

28. The method of claim 25, wherein the time-domain representation is a time series.

29. The method of claim 23, wherein the representation is a frequency domain representation.

30. The method of claim 23, further comprising taking one or more actions based on the loop stability measure.

31. A closed-loop neural stimulation system comprising:
a plurality of electrodes including one or more stimulation electrodes and one or more measurement electrodes;
a stimulus source configured to provide neural stimuli to be delivered via the one or more stimulation electrodes to a neural pathway of a patient in order to evoke neural responses on the neural pathway;
measurement circuitry configured to capture signal windows sensed on the neural pathway via the one or more measurement electrodes subsequent to respective neural stimuli;
a control unit configured to:
control the stimulus source to provide a neural stimulus according to a stimulus intensity parameter;
measure an intensity of the evoked neural response in the captured signal window subsequent to the provided neural stimulus;
determine a feedback variable from the measured intensity of the evoked neural response;
implement a feedback loop by using the feedback variable to control the stimulus intensity parameter so as to maintain the feedback variable at a target value; and
a processor configured to:
convert a loop variable of the feedback loop to a representation, wherein the representation is one of a time-domain representation and a frequency-domain representation; and
analyse the representation to determine a loop stability measure of the feedback loop.

32. The system of claim 31, further comprising an external device in communication with the control unit.

33. The system of claim 32, wherein the processor is part of the external device.

34. The system of claim 33, wherein the external device further comprises a display.

35. The system of claim 34, wherein the processor is configured to render a user interface on the display.

36. The system of claim 35, wherein the user interface comprises a control configured to allow a user to select the loop variable from among a plurality of candidate loop variables.

37. The system of claim 35, wherein the user interface comprises a control configured to allow a user to select a domain of the representation from among a plurality of candidate domains.

38. The system of claim 35, wherein the processor is further configured to display the loop stability measure on the user interface.

39. The system of claim 35, wherein the processor is further configured to display the representation on the user interface.
